(19) United States
(12) Patent Application Publication
(10) Pub. No.: US 2007/0259365 A1
(43) Pub. Date: Nov. 8, 2007

(54) Multifunctional Oligomer Probe Array and Method of Manufacturing the Same

(75) Inventors: Jung-hwan Hah, Hwaseong-si (KR); Sung-min Chi, Hwaseong-si (KR); Kyoung-seon Kim, Suwon-si (KR); Won-sun Kim, Suwon-si (KR)

Correspondence Address: F. CHAU & ASSOCIATES, LLC, 130 WOODBURY ROAD, WOODBURY, NY, 11797, US

(73) Assignee: Samsung Electronics Co., Ltd., Suwon-si (KR)

(21) Appl. No.: 11/743,477

(22) Filed: May 2, 2007

(30) Foreign Application Priority Data
May 2, 2006 (KR) 10-2006-0039717

(51) Int. Cl. C12Q 1/68; C07H 21/04; C12M 3/00

(52) U.S. Cl. 435/6; 435/287.2; 536/24.3; 977/924

(57) ABSTRACT

A multifunctional oligomer probe array capable of simultaneously performing different analyses such as gene expression profiling and genotyping includes a substrate, a first array region, a second array region, and a column spacer. The first array region has a plurality of first probe cell active regions, one or more oligomer probes, and a first probe cell defining region, wherein the plurality of first probe cell active regions are disposed on or in the substrate. The second array region has a plurality of second probe cell active regions, one or more oligomer probes, and a second probe cell defining region, wherein the plurality of second probe cell active regions are disposed on or in the substrate. The column spacer prevents cross-talk between a target sample applied to the first array region and another target sample applied to the second array region.

This application claims priority from Korean Patent Application No. 10-2006-0039717, filed on May 2, 2006, in the Korean Intellectual Property Office, the contents of which are herein incorporated by reference in their entirety.

### BACKGROUND OF THE INVENTION

1. Field of the Invention

The present disclosure is directed to an oligomer probe array, and more particularly, to a multifunctional oligomer probe array capable of simultaneously performing different analyses and a method of manufacturing the same.

2. Description of the Related Art

Oligomer probe arrays are tools that have been widely used in gene expression profiling, genotyping through detection of mutation or polymorphism such as Single-Nucleotide Polymorphism (SNP), protein or peptide assays, potential drug screening, development and preparation of novel drugs, etc.

Biological sample analyses using oligonucleotide probe assays, which are a type of oligomer probe arrays, are generally classified into gene expression profiling and genotyping. The gene expression profiling and the genotyping are different to each other from the viewpoints of importance and use. Thus, it is necessary to simultaneously perform the gene expression profiling and the genotyping for a single organism.

However, currently widely available oligomer probe arrays have been designed to implement only a single analysis. Thus, in order to simultaneously perform several different analyses, e.g., gene expression profiling and genotyping, the use of a plurality of oligomer probe arrays is needed, thereby causing a cost increase, resulting in a significant reduction in analysis efficiency.

### SUMMARY OF THE INVENTION

Embodiments of the present invention provide an oligomer probe array capable of simultaneously performing two or more analyses, including two or more different array regions in which no cross-talk between target samples occurs According to an embodiment of the present invention, a multifunctional oligomer probe array includes a substrate, a first array region having a plurality of first probe cell active regions, one or more oligomer probes, and a first probe cell defining region, wherein the plurality of first probe cell active regions are disposed on or in the substrate, each of which is coupled with oligomer probes having the same sequence, which is different to the sequences of oligomer probes coupled with the other first probe cell active regions, and the first probe cell defining region defines the first probe cell active regions and has no surface functional group for coupling with the oligomer probes; a second array region having a plurality of second probe cell active regions, one or more oligomer probes, and a second probe cell defining region, wherein the plurality of second probe cell active regions are disposed on or in the substrate, each of which is coupled with oligomer probes having the same sequence, which is different to the sequences of oligomer probes coupled to the other second probe cell active regions, and the second probe cell defining region defines the second probe cell active regions and has no surface functional group for coupling with the oligomer probes; and a column spacer for preventing cross-talk between a target sample applied to the first array region and another target sample applied to the second array region.

According to an embodiment of the present invention, a method of manufacturing a multifunctional oligomer probe array includes providing a substrate including a first array region and a second array region, wherein the first array region has a plurality of first probe cell active regions, one or more oligomer probes, and a first probe cell defining region, wherein the plurality of first probe cell active regions are disposed on or in the substrate, each of which is coupled with oligomer probes having the same sequence, which is different to the sequences of oligomer probes coupled with the other first probe cell active regions, and the first probe cell defining region defines the first probe cell active regions and has no surface functional group for coupling with the oligomer probes, and wherein the second array region has a plurality of second probe cell active regions, one or more oligomer probes, and a second probe cell defining region, wherein the plurality of second probe cell active regions are disposed on or in the substrate, each of which is coupled with oligomer probes having the same sequence, which is different to the sequences of oligomer probes coupled to the other second probe cell active regions, and the second probe cell defining region defines the second probe cell active regions and has no surface functional group for coupling with the oligomer probes; and attaching, on the substrate a column spacer for preventing cross-talk between a target sample applied to the first array region and another target sample applied to the second array region.

According to another embodiment of the present invention, a method of manufacturing a multifunctional oligomer probe array includes providing a substrate; forming a column spacer, which defines a first array region and a second array region by etching the substrate, and which prevents cross-talk between a target sample applied to the first array region and another target sample applied to the second array region; forming a plurality of first probe cell active regions defined by a first probe cell defining region on or in the first array region of the substrate, forming a plurality of second probe cell active regions defined by a first probe cell defining region on or in the second array region of the substrate, wherein each of the first probe cell defining region and the second probe cell defining region has no surface functional group for coupling with one or more oligomer probes; and coupling the plurality of first probe cell active regions and the second probe cell active regions with oligomer probes.

### BRIEF DESCRIPTION OF THE DRAWINGS

The above and other features of embodiments of the present invention will become more apparent by describing in detail exemplary embodiments thereof with reference to the attached drawings.

FIGS. 1A through 1C are sectional views illustrating multifunctional oligomer probe arrays including probe cell active regions patterned on a substrate according to an embodiment of the present invention.

FIGS. 2A through 2C are sectional views illustrating multifunctional oligomer probe arrays including probe cell active regions which are formed from LOCOS (LOCal Oxidation of Silicon) oxide films formed by local oxidation of a substrate according to an embodiment of the present invention.

FIGS. 3A through 3C are sectional views illustrating multifunctional oligomer probe arrays including trench-type probe cell active regions formed in a substrate according to an embodiment of the present invention.

FIG. 4 illustrates some modifications of the multifunctional oligomer probe arrays according to an embodiment of the invention.

FIG. 5 illustrates another modification of the multifunctional oligomer probe arrays, according to an embodiment of the invention.

FIG. 6 illustrates further modififications of the multifunctional oligomer probe arrays, according to an embodiment of the invention.

FIGS. 7A and 7B are sectional views of intermediate structures illustrating a method of manufacturing a multifunctional oligomer probe array according to an embodiment of the invention.

FIGS. 8A and 8B are sectional views of intermediate structures illustrating a method of manufacturing a multifunctional oligomer probe array according to an embodiment of the invention.

### DETAILED DESCRIPTION OF EXEMPLARY EMBODIMENTS

Features of the present invention and methods of accomplishing the same may be understood more readily by reference to the following detailed description of exemplary embodiments and the accompanying drawings. The present invention may, however, be embodied in many different forms and should not be construed as being limited to the embodiments set forth herein.

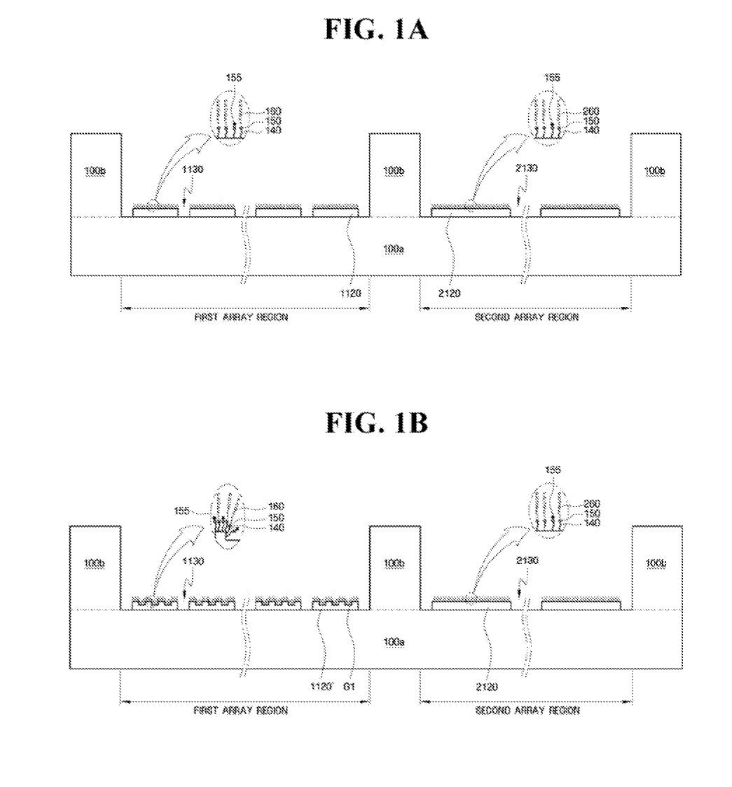

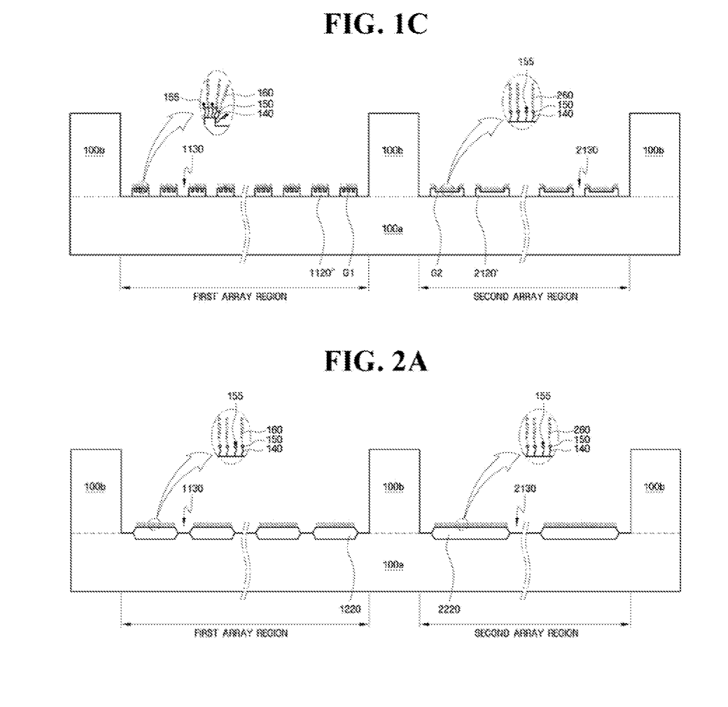

FIGS. 1A through 3C are sectional views illustrating various embodiments of multifunctional oligomer probe arrays.

Multifunctional oligomer probe arrays according to an embodiment of the present invention include first array regions and second array regions in which different analyses, e.g., genotyping and gene expression profiling can be respectively performed.

Gene expression profiling is a gene expression analysis of an organism, a tissue, etc. using transcript profiling. Thus, gene expression profiling results may be changed according to the type of an organism, the type of a tissue in even the same organism, or a biological state in even in the same tissue. That is, in the gene expression profiling, the gene expression profiles of a normal organism and a variant organism (e.g., an organism developing a disease or specific vital activity) are compared and analyzed.

Genotyping is determination of the genotypes of organisms, and the genotyping results of the same organisms are consistent regardless of the type and state of a tissue. That is, the genotyping is used to determined the differences of the inherited and genetic characters of several different organisms. Generally, individuals of the same species have similar genomic sequences, but show some sequence variations. Individuals have different characters due to the sequence variations. For example, the nucleotide sequences of a human population of 6 billion are 99.9% identical and differ from each other by only 0.1%. The remaining 0.1% is responsible for the differences among the humans. Thus, the genotyping of human genomes is analysis of the nucleotide sequence divergence of 0.1% to determine the differences among races, a nucleotide sequence causing a genetic disease, etc. For example, 1.42 million Single-Nucleotide Polymorphisms (SNPs) have been identified in the human genome. Various combinations of the SNPs are represented by haplotypes which are minimum units for the expression of different characters among individuals in a human population of 6 billion. That is, the gene expression profiling and the genotyping have different biological meanings, and thus, it is useful to simultaneously perform gene expression profiling and genotyping for an organism.

In this regard, referring to FIGS. 1A through 3C, multifunctional oligomer probe arrays according to an embodiment of the present invention include first array regions 101 and second array regions 102 capable of simultaneously performing respective different analyses.

Referring to FIGS. 1A through 3C, an oligomer probe array includes column spacer 100b for preventing cross-talk between target samples applied to first array regions 101 and second array regions 102. In order to effectively prevent cross-talk between target samples, the column spacers 100b may have a height of 100 to 500 μm and a width of 1 to 20 mm.

The column spacers 100b may be integral-type column spacers formed integrally with a substrate 100a or attachment-type column spacers attached to the substrate 100a. The integral-type column spacers may be formed by etching the substrate 100a. Although FIGS. 1A through 3C illustrate that the column spacers 100b have line profile, the shapes of the column spacers 100b may be diversely changed.

The number of probes required for the analysis of the various genetic information of the human genome and the design rules of regions coupling with the probes are summarized in Table 1 below.

TABLE 1

| Genetic | The number | | | |
|---|---|---|---|---|
| information | of probes | Design rule | | Remark |
| Gene | ˜34,000 | >10 | μm | DNA strand of a |
| | | | | specific position in |
| | | | | a chromosome |
| Exon | ˜450,000 | 5 | μm | Minimum unit for |
| | | | | expression of |
| | | | | genetic information |
| Haplotype | 450,000˜1,000,000 | 5 | μm | Minimum unit for |
| | | | | expression of |
| | | | | different characters |
| | | | | among individuals |
| SNP | 1,420,000 | <5 | μm | Variation |
| Nucleotide | 3,000,000,000 | <1 | μm | Minimum unit of DNA |

As shown in Table 1 above, different design rules are applied according to desired information. When a design rule of more than 10 μm is employed, genetic information on a DNA strand of a specific position in a chromosome can be acquired. On the other hand, when a design rule of 5 μm or less is employed, various analyses such as analysis of exons which are minimum units for the expression of genetic information, analysis of haplotypes which are minimum units for the expression of different characters among individuals, and SNP analysis can be performed. When a design rule of less than 1 μm is employed, the sequence analysis of nucleotides which are minimum units of DNA is possible.

In this regard, in the oligomer probe arrays illustrated in FIGS. 1A through 3C, design rules of first probe cell active regions 1120, 1120′, 1120″, 1220, 1220′, 1220″, 1320, 1320′, and 1320″ and each of corresponding second probe cell active regions 2120, 2120′, 2220, 2220′, 2320, and 2320′ can be diversely selected according to the types of analyses to be executed in the oligomer probe arrays.

FIGS. 1A through 3C illustrate that the design rules of the first probe cell active regions 1120, 1120′, 1120″, 1220, 1220′, 1220″, 1320, 1320′, and 1320″ constituting the first array regions are smaller than those of the second probe array regions 2120, 2120′, 2220, 2220′, 2320, and 2320′ constituting the second array regions. However, it should be understood that the design rules of the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ can be diversely changed when needed.

The multifunctional oligomer probe arrays illustrated in FIGS. 1A through 1C include a substrate 100a, first array regions including first probe cell active regions 1120, 1120′, and 1120″ patterned on the substrate 100a and cell defining region 1130 defining the first probe cell active regions 1120, 1120′, and 1120″ and having no surface functional group for coupling with oligomer probes 160; and second array regions including second probe cell active regions 2120, 2120′ patterned on the substrate 100a and cell defining regions 2130 defining the second probe cell active regions 2120, 2120′ and having no surface functional group for coupling with oligomer probes 260.

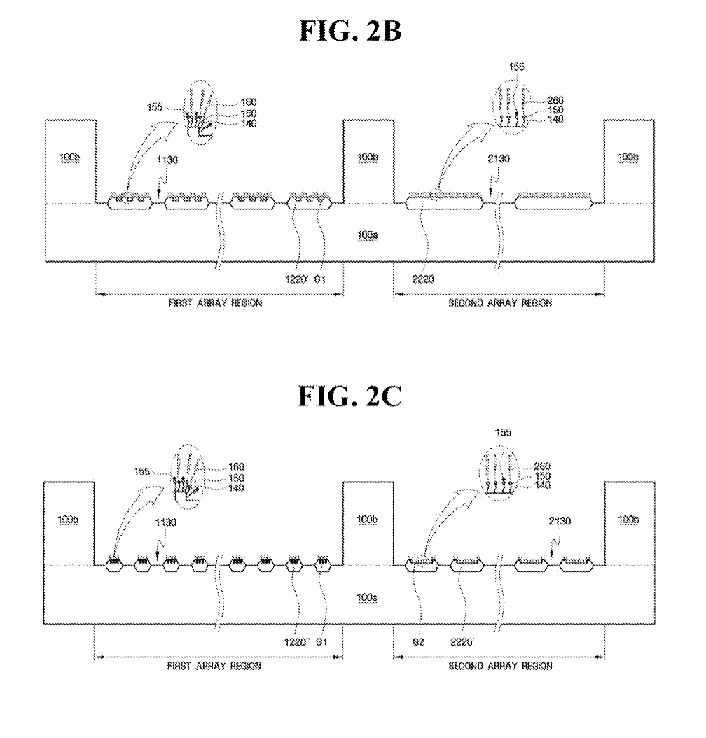

The multifunctional oligomer probe arrays illustrated in FIGS. 2A through 2C include a substrate 100a; first array regions including first probe cell active regions 1220, 1220′, and 1220″ which are formed from LOCOS (LOCal Oxidation of Silicon) oxide films obtained by local oxidation of the substrate 100a and cell defining regions 1130 defining the first probe cell active regions 1220, 1220′, and 1220″ and having no surface functional group for coupling with oligomer probes 160; and second array regions including second probe cell active regions 2220, 2220′ which are formed from LOCOS oxide films obtained by local oxidation of the substrate 100a and cell defining regions 2130 defining the second probe cell active regions 2220, 2220′ and having no surface functional group for coupling with oligomer probes 260.

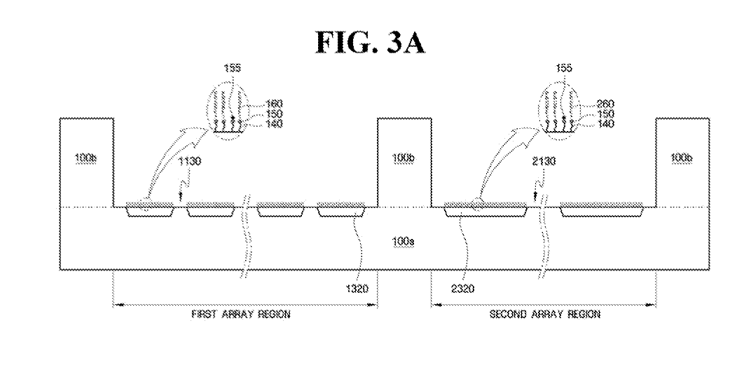

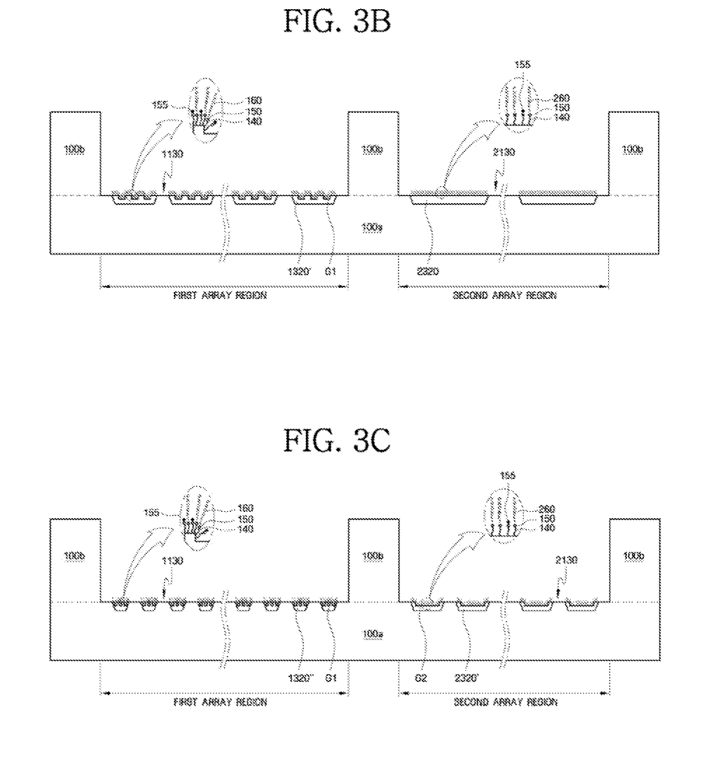

The multifunctional oligomer probe arrays illustrated in FIGS. 3A through 3C include a substrate 100a; first array regions including first trench-type probe cell active regions 1320, 1320′, and 1320″ formed in the substrate 100a and cell defining regions 1130 defining the first trench-type probe cell active regions 1320, 1320′, and 1320″ and having no surface functional group for coupling with oligomer probes 160, and second array regions including second trench-type probe cell active regions 2320, 2320′ formed in the substrate 100a and cell defining regions 2130 defining the second trench-type probe cell active regions 2320, 2320′ and having no surface functional group for coupling with oligomer probes 260.

As used herein, the term “oligomer” is a low-molecular weight polymer molecule consisting of two or more covalently bound monomers. Oligomers have a molecular weight of about 1,000 or less but the present invention is not limited thereto. The oligomer may include about 2-500 monomers, or 5-30 monomers. The monomers may be nucleosides, nucleotides, amino acids, peptides, etc., according to the type of probes. In the present invention, previously synthesized oligomer probes may be coupled to active regions, or oligomer probes may be synthesized on active regions by in-situ photolithography.

As used herein, the terms “nucleosides” and “nucleotides” include not only known purine and pyrimidine bases, but also methylated purines or pyrimidines, acylated purines or pyrimidines, etc. Furthermore, the “nucleosides” and “nucleotides” include not only known (deoxy)ribose, but also a modified sugar which contains a substitution of a halogen atom or an aliphatic group for at least one hydroxyl group or is functionalized with ether, amine, or the like.

The substrate 100a may be made of a material capable of minimizing or substantially preventing unwanted non-specific bonds during hybridization and of transmitting visible and/or UV light. The substrate 100 may be a flexible or rigid substrate. When a flexible substrate is used as the substrate 100, the substrate 100 may be a nylon membrane, a nitrocellulose membrane, a plastic film, etc. When a rigid substrate is used as the substrate 100, the substrate 100 may be a silicone substrate, a transparent glass (e.g., soda-lime glass) substrate, etc. The use of a silicone substrate or a transparent glass substrate as the substrate 100 is useful in that non-specific binding rarely occurs during hybridization. Furthermore, a transparent glass substrate is transparent to visible light and/or UV light, and thus, is useful in detection of a fluorescent material. In addition, when a silicone substrate or a transparent glass substrate is used as the substrate 100, it is possible to employ various thin film formation processes and photolithography processes that have been well established and stably applied in the fabrication of semiconductor devices or liquid crystal display (LCD) panels.

The first probe cell active regions 1120, 1120′, 1120″, 1220, 1220′, 1220″, 1320, 1320′, and 1320″ and the second probe cell active regions 2120, 2120′, 2220, 2220′, 2320, and 2320′ may be made of a material that is substantially stable against hydrolysis upon hybridization assay, e.g., upon contacting with a pH 6-9 phosphate or Tris buffer. Accordingly, the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ may be made of a silicon oxide layer such as a plasma enhanced-TEOS (PE-TEOS) layer, a high density plasma (HDP) oxide layer, a P—SiH₄ oxide layer or a thermal oxide layer; silicate such as hafnium silicate or zirconium silicate; a silicon nitride layer; a silicon oxynitride layer; a metal oxynitride layer such as a hafnium oxynitride layer or a zirconium oxynitride layer; a metal oxide layer such as a titanium oxide layer, a tantalum oxide layer, an aluminum oxide layer, a hafnium oxide layer, a zirconium oxide layer or an indium tin oxide (ITO) layer; polyimide; polyamine; a metal such as gold, silver, copper or palladium; or a polymer such as polystyrene, polyacrylate or polyvinyl.

In the multifunctional oligomer probe arrays illustrated in FIGS. 1A through 3C, functional groups 150 capable of coupling with the oligomer probes 160 and 260 or monomers for in-situ synthesis of the oligomer probes 160 and 260, hereinafter referred to as “functional groups 150 capable of coupling with the oligomer probes 160 and 260”, are present on surfaces of the first probe cell active regions 1120, 1120′, 1120″, 1220, 1220′, 1220″, 1320, 1320′, and 1320″ and the second probe cell active regions 2120, 2120′, 2220, 2220′, 2320, and 2320′, but absent on surfaces of the probe cell defining regions 1130 and 2130.

The functional groups 150 are groups that can be used as starting points for organic synthesis. That is, the functional groups 150 are groups capable of coupling with, e.g., covalently or non-covalently binding with, the previously synthesized oligomer probes 160, 260 or the monomers (e.g., nucleosides, nucleotides, amino acids, or peptides) for in-situ synthesis of the oligomer probes 160, 260. The functional groups 150 are not limited to any particular functional groups, provided that they can be coupled to the oligomer probes 160, 260 or the monomers for in-situ synthesis of the oligomer probes 160, 260. Examples of the functional groups 150 include hydroxyl groups, aldehyde groups, carboxyl groups, amino groups, amide groups, thiol groups, halo groups, and sulfonate groups.

Thus, the oligomer probes 160 and 260 are coupled to the surfaces of the first probe cell active regions 1120, 1120′, 1120″, 1220, 1220′, 1220″, 1320, 1320′, and 1320″ and the second probe cell active regions 2120, 2120′, 2220, 2220′, 2320, and 2320′, but not to the probe cell defining regions 1130 and 2130. Therefore, a SNR can be increased in oligomer probe array-based analyses, thereby increasing analysis accuracy.

FIGS. 1A through 3C illustrate that the functional groups 150 capable of coupling with, e.g., covalently binding with the oligomer probes 160 and 260, are connected to the surfaces of the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ via linkers 140.

However, in a case where a material constituting the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ includes the functional groups 150, the linkers 140 may be omitted. Even in a case where the functional groups 150 are not included in a material constituting the first probe cell active regions 1120, 1120′, 1120″, 1220, 1220′, 1220″, 1320, 1320′, and 1320″ and the second probe cell active regions 2120, 2120′, 2220, 2220′, 2320, and 2320′, they can be directly provided on the surfaces of the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ by a surface treatment. The surface treatment may be ozonolysis, acid treatment, base treatment, etc. That is, the formation of the linkers 140 is optional.

The linkers 140, when used, serve to facilitate a free interaction (e.g., hybridization) between the oligomer probes 160 and 260 and target samples. Thus, the linkers 140 may have a sufficient length to ensure free probe-target interaction. The molecular length of the linkers 140 may be 6-50 atoms, but the present invention is not limited thereto.

The linkers 140 may be made of a material including coupling groups capable of coupling with the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ and the functional groups 150 capable of coupling with monomers for in-situ photolithographic synthesis of the oligomer probes 160 and 260. The functional groups 150 may be protected with protecting groups. Furthermore, the linkers 140 coupled to the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ before the in-situ synthesis of the oligomer probes 160 and 260 may be attached with protecting groups for the purpose of storage. As used herein, the term “protecting groups” refer to groups used to render chemically reactive moieties inactive until deprotection occurs, and the term “deprotection” refers to the removal of the protecting groups to render the inactivated moieties chemically reactive. For example, acid-labile or photolabile protecting groups may be attached to the functional groups 150 of the linkers 140 to protect the functional groups 150. The protecting groups may be removed before monomers used for in-situ photolithographic synthesis or the oligomer probes 160 and 260 are coupled to the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ to expose the functional groups 150.

When the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ are made of silicon oxide, silicate, or silicon oxynitride, the coupling groups of the linkers 140 may include silicone groups capable of producing siloxane (Si—O) bonds with Si(OH) groups on surfaces of the first probe cell active regions 1120, 1120′, 1120″, 1220, 1220′, 1220″, 1320, 1320′, and 1320″ and the second probe cell active regions 2120, 2120′, 2220, 2220′, 2320, and 2320′. Examples of the coupling groups of the linkers 140 include —Si(OMe)₃, —SiMe(OMe)₂, —SiMeCl₂, —SiMe(OEt)₂, —SiCl₃, —Si(OEt)₃, and the like. Examples of the material including the functional group 150 and containing a silicon group capable of creating a siloxane bond include N-(3-(triethoxysilyl)-propyl)-4-hydroxybutyramide, N,N-bis(hydroxyethyl)aminopropyl-triethoxysilane, acetoxypropyl-triethoxysilane, 3-glycidoxy propyltrimethoxysilane, silicone compounds disclosed in International Patent Publication No. WO 00/21967, issued as U.S. Pat. No. 6,262,216, and the like, the disclosures of which are hereby incorporated by reference as fully set forth herein.

When the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ are made of metal oxide, the coupling groups of the linkers 140 may include metal alkoxide groups or metal carboxylate groups.

When the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ are made of silicon nitride, silicon oxynitride, metal oxynitride, polyimide, or polyamine, the coupling groups of the linkers 140 may include anhydride groups, acid chloride groups, alkyl halide groups, or chlorocarbonate groups.

When the first probe cell active regions 1120, 1120′, 1120″, 1220, 1220′, 1220″, 1320, 1320′, and 1330″ and the second probe cell active regions 2120, 2120′, 2220, 2220′, 2320, and 2320′ are made of metal, the coupling groups of the linkers 140 may include sulfide groups, selenide groups, arsenide groups, telluride groups, or antimonide groups.

When the first probe cell active regions 1120, 1120′, 1120″, 1220, 1220′, 1220″, 1320, 1320′, and 1320″ and the second probe cell active regions 2120, 2120′, 2220, 2220′, 2320, and 2320′ are made of a polymer, the coupling groups of the linkers 140 may include acrylic groups, styryl groups, or vinyl groups.

FIGS. 1A, 2A, and 3A illustrate that the first probe cell active regions 1120, 1220, and 1320 and the second probe cell active regions 2120, 2220, and 2320 have substantially flat upper surfaces.

FIGS. 1B, 2B, and 3B illustrate that the first probe cell active regions 1120′, 1220′, and 1320′ have three-dimensional surfaces. Thus, an area capable of coupling with the oligomer probes 160, 260 can be increased, and thus, the number of the oligomer probes 160, 260 coupled to the first probe cell active regions 1120′, 1220′, and 1320′ can be increased, compared to the first probe cell active regions 1120, 1220, and 1320 illustrated in FIGS. 1A, 2A, and 3A having the same design rule. Therefore, even when a reduced design rule is employed, desired detection sensitivity can be ensured. The three-dimensional surfaces of the first probe cell active regions 1120′, 1220′, and 1320′ are defined by grooves G1 formed in the first probe cell active regions 1120′, 1220′, and 1320′. However, it should be understood that structures capable of defining a three-dimensional surface are not limited to the grooves G1.

FIGS. 1C, 2C, and 3C illustrate that the first probe cell active regions 1120″, 1220″, and 1320″ and the second probe cell active regions 2120′, 2220′ and 2320′ have three-dimensional surfaces by first grooves G1 and second grooves G2. In this case, although the design rules of the first array regions and the second array regions are remarkably reduced, the number of the oligomer probes 160 and 260 coupled to the first probe cell active regions 1120″, 1220″, and 1320″ and the second probe cell active regions 2120′, 2220′ and 2320′ can be increased due to the three-dimensional surfaces, thereby ensuring desired detection sensitivity. Even in a case where the same design rule is applied to the first array regions and the second array regions, multifunctional probe arrays can be embodied by changing the shapes of the first and second grooves G1 and G2.

In the multifunctional oligomer probe arrays illustrated in FIGS. 1A through 3C, the probe cell defining regions 1130 and 2130 having no functional group for coupling with the oligomer probes 160 and 260 may be exposed surface regions of a silicone substrate or a transparent substrate.

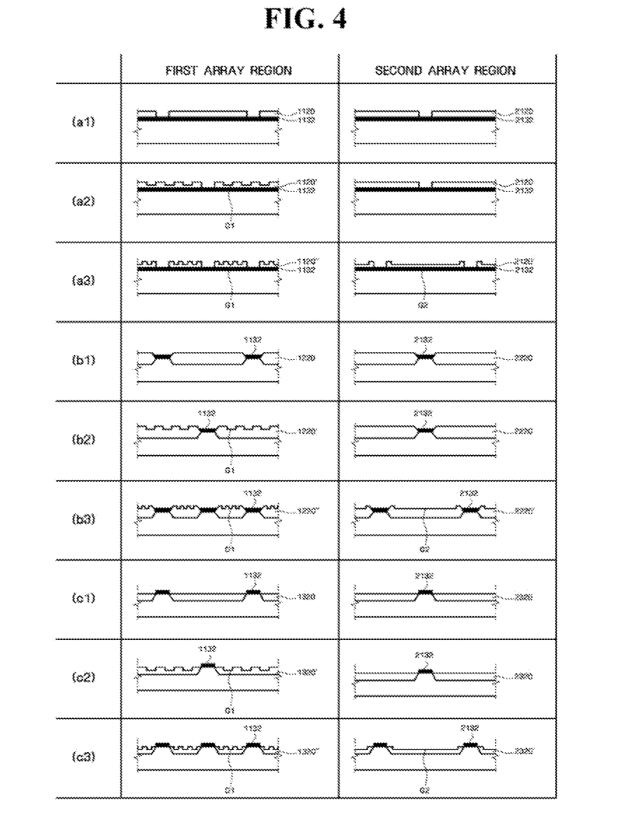

In some modifications of the multifunctional oligomer probe arrays illustrated in FIGS. 1A through 3C, as shown in FIG. 4, the probe cell defining regions 1130 and 2130 may be exposed regions of blocking films 1132 and 2132 formed on the entire surfaces of the substrate 100a or blocking films 1132 and 2132 formed on exposed regions of the substrate 100a through the first and second probe cell active regions 1220, 1220′, 1220″, 2220, 2220′, 1320, 1320′, 1320″, 2320, and 2320′. The blocking films 1132 and 2132 may be made of fluorine-containing fluoride such as fluorosilane. Also, the blocking films 1132 and 2132 may be silicide films, polysilicone films, or epitaxial films of Si or SiGe.

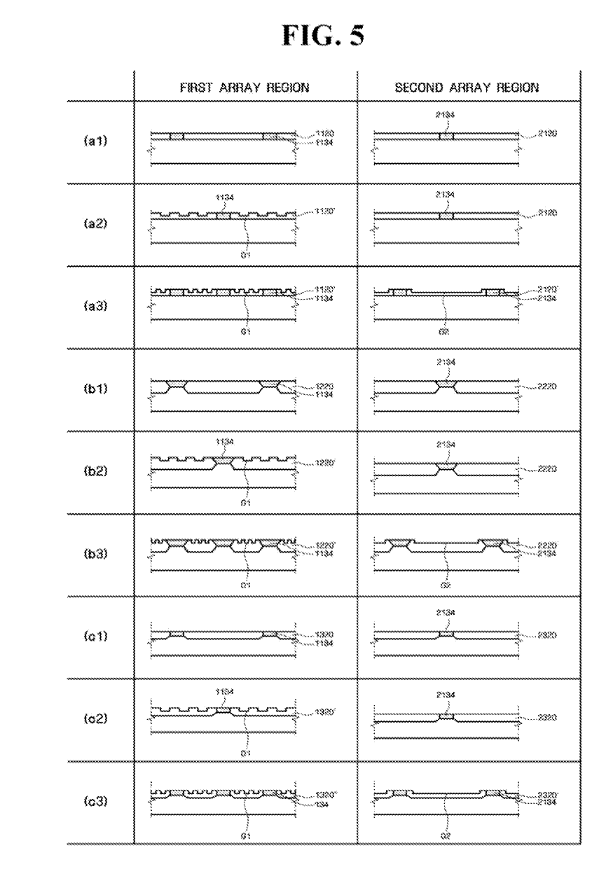

In some further modifications of the multifunctional oligomer probe arrays illustrated in FIGS. 1A through 3C, as shown in FIG. 5, the probe cell defining regions 1130 and 2130 may be fillers 1134 and 2134 that have characteristics preventing the coupling of the oligomer probes 160 and 260 and are filled in areas defined between the first probe cell active regions 1120, 1120′, 1120″, 1220, 1220′, 1220″, 1320, 1320′, 1320″, and in areas defined between the second probe cell regions 2120, 2120′, 2220, 2220′, 2320, and 2320′. The fillers 1134 and 2134 may also be made of fluorine-containing fluoride, polysilicone, etc.

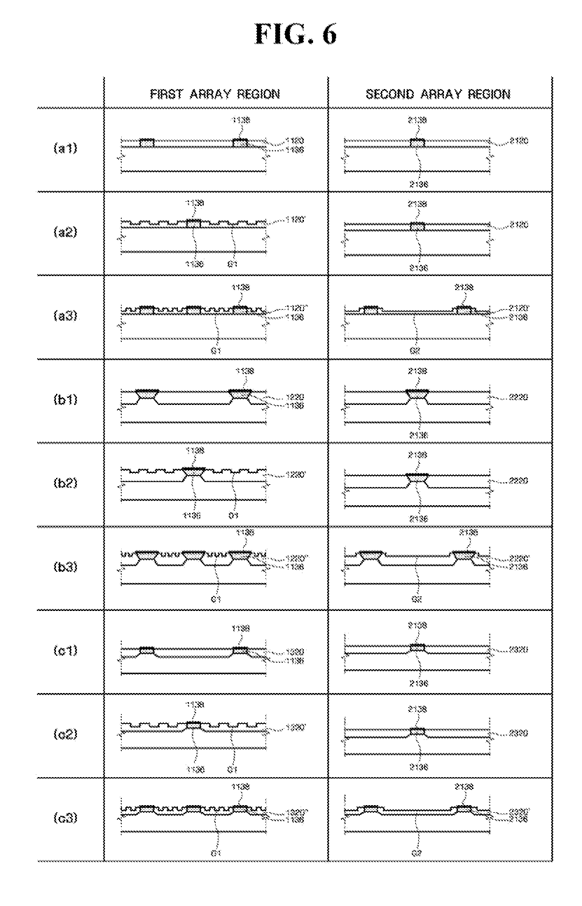

In some further modifications of the multifunctional oligomer probe arrays illustrated in FIGS. 1A through 3C, as shown in FIG. 6, the probe cell defining regions 1130 and 2130 may be comprised of fillers 1136 and 2136 filled in areas defined between the first probe cell active regions 1120, 1120′, 1120″, 1220, 1220′, 1220″, 1320, 1320′, 1320″, and the second probe cell active regions 2120, 2120′, 2220, 2220′, 2320, and 2320′ and blocking films 1138 and 2138 formed on the fillers 1136 and 2136. In this case, it is not necessarily required that the fillers 1136 and 2136 have characteristics preventing the coupling of the oligomer probes 160 and 260.

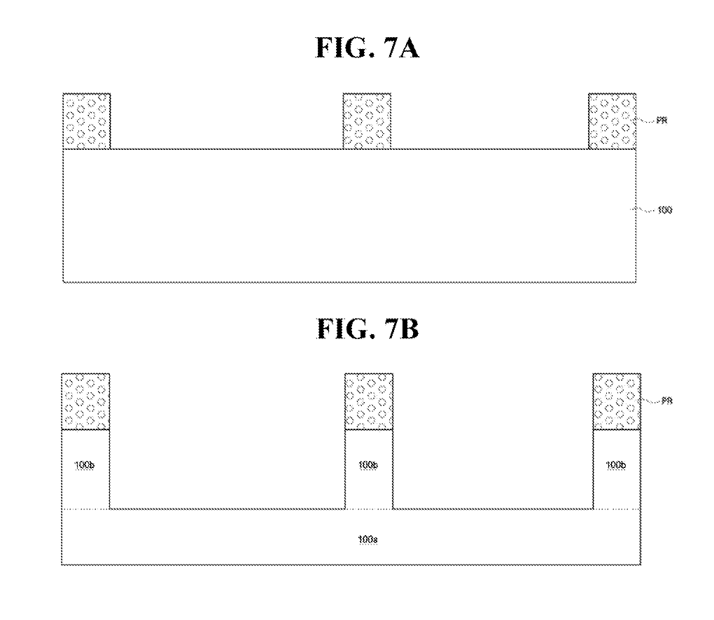

FIGS. 7A and 7B are sectional views of intermediate structures illustrating a method of manufacturing a multifunctional oligomer probe array according to an embodiment of the present invention.

Referring to FIG. 7A, a substrate 100 in which a first array region and a second array region are defined is prepared. Then, a photoresist pattern PR defining column spacers for partitioning the first array region and the second array region is formed on the substrate 100.

Referring to FIG. 7B, the substrate 100 is etched using the photoresist pattern PR as an etching mask to form column spacers 100b of 100-500 μm in height and 1-20 mm in width and to provide a substrate 100a on which probe cell active regions are to be formed.

The subsequent processes of forming probe cell active regions in the first array region and the second array region are fully disclosed in Korean Patent Application No. 2006-0039716, entitled “Oligomer Probe Array with Improved Signal-to-Noise Ratio and Method of Manufacturing the Same”, and Korean Patent Application No. 2006-0039713, entitled “Oligomer Probe Array with Improved Signal-to-Noise Ratio and Detection Sensitivity and Method of Manufacturing the Same”, filed by the present applicant, the disclosures of which are herein incorporated by reference in their entireties.

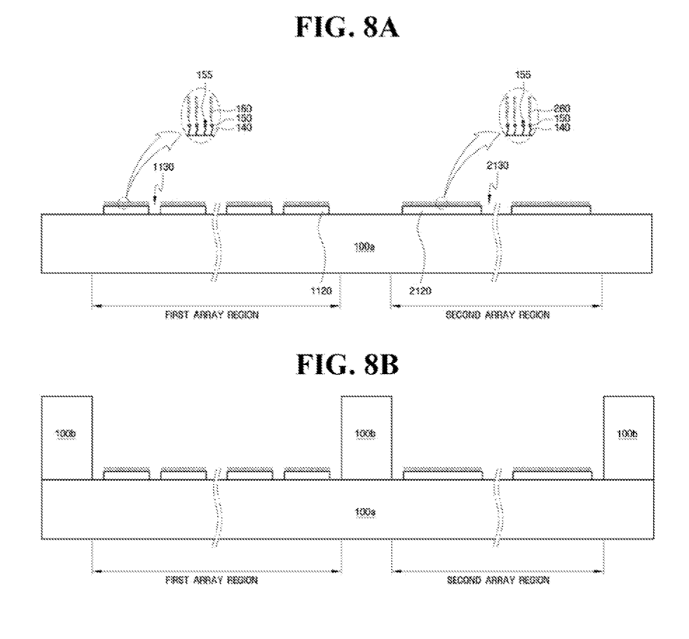

FIGS. 8A and 8B are sectional views of intermediate structures illustrating a method of manufacturing an oligomer probe array according to an embodiment of the invention illustrated in FIG. 1A.

Referring to FIG. 8A, oligomer probes 160, 260 are completed on each of a first array region 101 and a second array region 102 of a substrate 100a.

Referring to FIGS. 8A and 8B, column spacers 100b of 100-500 μm in height and 1-20 mm in width are attached to a surface of the substrate 100a to complete a multifunctional oligomer probe array.

Hereinafter, the present invention will be described more specifically with reference to the following experimental examples.

### EXPERIMENTAL EXAMPLE 1

PE-TEOS films were formed to a thickness of 500 nm on silicone wafers using a Chemical Vapor Deposition (CVD) process. Then, photoresist films were formed to a thickness of 3.0 μm on the resultant structures using a spin-coating process and baked at 100° C. for 60 seconds. Then, the photoresist films were exposed to light in a 365 nm-wavelength projection exposure machine and then developed with a 2.38% TetraMethylAmmonium Hydroxide (TMAH) solution to form checkerboard type photoresist patterns so that the underlying PE-TEOS films were exposed in the form of a plurality of intersecting stripes. The PE-TEOS films were etched using the photoresist patterns as etching masks to form PE-TEOS film patterns with a width of 5 μm in first array regions and PE-TEOS film patterns with a width of 10 μm in second array regions. Then, polysilicone was coated on the entire surfaces of the resultant structures using a CVD process and planarized using a Chemical Mechanical Polishing (CMP) process to form fillers having characteristics preventing the coupling of oligomer probes and being filled in areas defined between the PE-TEOS film patterns.

Next, the PE-TEOS film patterns were coated with bis(hydroxyethyl)aminopropyltriethoxysilane, treated with an acetonitrile solution containing amidite-activated NNPOC-tetraethyleneglycol and tetrazole (1:1) so that phosphoramidite was coupled to the PE-TEOS film patterns, and then acetyl-capped to thereby complete protected linker structures.

Next, in-situ photolithographic synthesis of oligonucleotide probes was performed on the PE-TEOS film patterns. For this, the PE-TEOS film patterns were exposed to light using a binary mask exposing predetermined PE-TEOS film patterns in a 365 nm-wavelength projection exposure machine with an energy of 1000 mJ/cm²for one minute to deprotect terminating functional groups of the linker structures. Then, the PE-TEOS film patterns were treated with an acetonitrile solution containing amidite-activated nucleotide and tetrazole (1:1) to achieve coupling of the protected nucleotide monomers to the deprotected linker structures, and then treated with a THF solution (acetic anhydride (Ac20)/pyridine (py)/methylimidazole=1:1:1) and a 0.02 M iodine-THF solution to perform capping and oxidation.

The above-described deprotection, coupling, capping, and oxidation processes were repeated to form oligonucleotide probes having complementary sequences to transcripts of full genome sequences, mRNAs, or specific regions of genes on probe cell active regions corresponding to the first array regions, and oligonucleotide probes having complementary sequences to polymorphic sites of full genome sequences or specific regions of upstream/downstream sequences on probe cell active regions corresponding to the second array regions. Then, column spacers having a width of 1 mm and a height of 100 μm were attached to boundary areas between the first array regions and the second array regions, to complete multifunctional oligonucleotide probe arrays having the first array regions for gene expression profiling and the second array regions for genotyping.

### EXPERIMENTAL EXAMPLE 2

Photoresist films were formed to a thickness of 1.2 μm on silicone wafers using a spin-coating process and baked at 100° C. for 60 seconds. Then, the photoresist films were exposed to light in a 365 nm-wavelength projection exposure machine and developed with a 2.38% TMAH solution to form photoresist patterns. Then, the silicone wafers were etched using the photoresist patterns as etching masks in CF₄ based plasma to form column spacers having a width of 1 mm and a height of 100 μm, thereby completing the substrate in which first array regions and second array regions were defined.

Next, formation of probe cell active regions, formation of linkers, and in-situ synthesis of oligonucleotide probes were performed in substantially the same manner as in Experimental Example 1 to complete multifunctional oligonucleotide probe arrays.

In multifunctional oligomer probe arrays according to embodiments of the present invention, different analyses, e.g., genotyping and gene expression profiling can be performed at the same time, thereby increasing analysis efficiency and greatly decreasing analysis costs.

Furthermore, functional groups capable of coupling with oligomer probes are present on surfaces of probe cell active regions, but absent on surfaces of probe cell defining regions. Therefore, oligomer probes are coupled to the probe cell active regions but not to the probe cell defining regions surrounding the probe cell active regions. As a result, a SNR can be increased in analyses using oligomer probe arrays, thereby increasing analysis accuracy.

In addition, in a case where the probe cell active regions have a three-dimensional surface, an area capable of coupling with oligomer probes can be increased, and thus, the number of oligomer probes capable of coupling with each probe cell active can be increased, compared to conventional oligomer probe arrays having the same design rule as the oligomer probe arrays of the present invention. Therefore, even when a reduced design rule is employed, desired detection sensitivity can be ensured.

What is claimed is:

1. A multifunctional oligomer probe array comprising:
a substrate;
a first array region having a plurality of first probe cell active regions, one or more oligomer probes, and a first probe cell defining region, wherein the plurality of first probe cell active regions are disposed on or in the substrate, each of which is coupled with oligomer probes having the same sequence, which is different to the sequences of oligomer probes coupled with the other first probe cell active regions, and the first probe cell defining region defines the first probe cell active regions and has no surface functional group for coupling with the oligomer probes;
a second array region having a plurality of second probe cell active regions, one or more oligomer probes, and a second probe cell defining region, wherein the plurality of second probe cell active regions are disposed on or in the substrate, each of which is coupled with oligomer probes having the same sequence, which is different to the sequences of oligomer probes coupled to the other second probe cell active regions, and the second probe cell defining region defines the second probe cell active regions and has no surface functional group for coupling with the oligomer probes; and
a column spacer for preventing cross-talk between a target sample applied to the first array region and another target sample applied to the second array region.

2. The multifunctional oligomer probe array of claim 1, wherein the first array region is used for gene expression profiling and the second array region is used for genotyping.

3. The multifunctional oligomer probe array of claim 1, wherein the column spacer is either an integral-type column spacer that is integrally formed with the substrate or an attachment-type column spacer that is attached to the substrate.

4. The multifunctional oligomer probe array of claim 3, wherein the column spacer has a height of 100 to 500 μm and a width of 1 to 20 mm.

5. The multifunctional oligomer probe array of claim 1, wherein each of he first and second probe cell active regions have either a flat surface or a three-dimensional surface.

6. The multifunctional oligomer probe array of claim 5, wherein the three-dimensional surface is produced by one or more groove formed in the first probe cell active regions and/or the second probe cell active regions.

7. The multifunctional oligomer probe array of claim 1, wherein the first and second probe cell active regions comprise surface functional groups capable of coupling with the oligomer probes, and
wherein some of the functional groups are coupled to the oligomer probes and the other functional groups are rendered inactive by capping.

8. The multifunctional oligomer probe array of claim 1, wherein each of the first and second probe cell active regions are either film patterns formed on the substrate, LOCOS oxide films formed by local oxidation of the substrate, or trench-type active regions filling trenches in the substrate.

9. The multifunctional oligomer probe array of claim 8, wherein each surface of the first and second probe cell defining regions is either an exposed surface of a silicone substrate or a transparent substrate.

10. The multifunctional oligomer probe array of claim 8, wherein each surface of the first and second probe cell defining regions is a surface of a blocking film that is disposed on an upper surface of the substrate and has characteristics preventing the coupling of the oligomer probes.

11. The multifunctional oligomer probe array of claim 8, wherein each surface of the first and second probe cell defining regions is a surface of a filler that is filled in an area defined between the first or second probe cell active regions and has characteristics preventing the coupling of the oligomer probes.

12. The multifunctional oligomer probe array of claim 8, wherein each surface of the first and second probe cell defining regions is a surface of a blocking film that is disposed on a filler filled in an area defined between the first or second probe cell active regions and has characteristics preventing the coupling of the oligomer probes.

13. The multifunctional oligomer probe array of claim 1, wherein the oligomer probes are coupled to the first and second probe cell active regions via linkers.

14. A method of manufacturing a multifunctional oligomer probe array, the method comprising:
providing a substrate including a first array region and a second array region, wherein the first array region has a plurality of first probe cell active regions, one or more oligomer probes, and a first probe cell defining region, wherein the plurality of first probe cell active regions are disposed on or in the substrate, each of which is coupled with oligomer probes having the same sequence, which is different to the sequences of oligomer probes coupled with the other first probe cell active regions, and the first probe cell defining region defines the first probe cell active regions and has no surface functional group for coupling with the oligomer probes, and wherein the second array region has a plurality of second probe cell active regions, one or more oligomer probes, and a second probe cell defining region, wherein the plurality of second probe cell active regions are disposed on or in the substrate, each of which is coupled with oligomer probes having the same sequence, which is different to the sequences of oligomer probes coupled to the other second probe cell active regions, and the second probe cell defining region defines the second probe cell active regions and has no surface functional group for coupling with the oligomer probes; and
attaching, on the substrate, a column spacer for preventing cross-talk between a target sample applied to the first array region and another target sample applied to the second array region.

15. The method of claim 14, wherein the first array region is used for gene expression profiling and the second array region is used for genotyping.

16. A method of manufacturing a multifunctional oligomer probe array, the method comprising:
providing a substrate;
forming a column spacer, which defines a first array region and a second array region by etching the substrate, and which prevents cross-talk between a target sample applied to the first array region and another target sample applied to the second array region;
forming a plurality of first probe cell active regions defined by a first probe cell defining region on or in the first array region of the substrate, forming a plurality of second probe cell active regions defined by a first probe cell defining region on or in the second array region of the substrate, wherein each of the first probe cell defining region and the second probe cell defining region has no surface functional group for coupling with one or more oligomer probes; and
coupling the plurality of first probe cell active regions and the second probe cell active regions with oligomer probes.

17. The method of claim 16, wherein the first array region is used for gene expression profiling and the second array region is used for genotyping.
